(19) United States
(12) Patent Application Publication
(10) Pub. No.: US 2022/0172085 A1
(43) Pub. Date: Jun. 2, 2022

(54) Methods and Systems to Account for Uncertainties from Missing Covariates in Generative Model Predictions

(71) Applicant: Unlearn.AI, Inc., San Francisco, CA (US)

(72) Inventors: Charles Kenneth Fisher, Truckee, CA (US); Jonathan Ryan Walsh, El Cerrito, CA (US); David Walsh, San Francisco, CA (US)

(73) Assignee: Unlearn.AI, Inc., San Francisco, CA (US)

(21) Appl. No.: 17/457,233

(22) Filed: Dec. 1, 2021

Related U.S. Application Data
(60) Provisional application No. 63/119,847, filed on Dec. 1, 2020.

(51) Int. Cl. G06N 5/04

(52) U.S. Cl. CPC G06N 5/048; G06F 17/10

(57) ABSTRACT

Systems and methods to account for uncertainties from missing covariates in generative model predictions. One embodiment includes a method for updating the values for uncertainty used in a generative model that is created using a set of known prognostically important baseline data. The method includes steps for determining a value, within the generative model, for the variance in outcome given the known prognostically important baseline data, wherein the steps include imputing values for a set of unknown prognostically important baseline data, and determining estimations for explained and unexplained variance in outcome for each subject when given both sets of data.

### CROSS-REFERENCE TO RELATED APPLICATIONS

The current application claims the benefit of and priority under 35 U.S.C. § 119(e) to U.S. Provisional Patent Application No. 63/119,847 entitled “Accounting for Uncertainties from Missing Baseline Data in Digital Twin Predictions” filed Dec. 1, 2020, the disclosure of which is hereby incorporated by reference in its entirety for all purposes.

### FIELD OF THE INVENTION

The present invention generally relates to defining uncertainty in generative predictive models and, more specifically, enabling these models to maintain accurate predictions in response to uncertainties that can result from missing or insubstantial baseline data.

### BACKGROUND

Generative models have various applications in a variety of fields. Traditionally, these models are trained using observations from a data distribution, while summary statistics are similarly useful in training or adjusting generative models. In responding to the predictions output by generative models, the degree of uncertainty will naturally have a substantial impact (e.g., in predicting clinical outcomes for patients with a disease, a physician's recommended treatment course may differ if a predictive model is confident). As such, generative model predictions in relation to a population will naturally be conditioned on the initial, or ‘baseline’, data available for that population. When baseline data is entirely (or predominantly) known, generative predictive models will be particularly robust. Conversely, when prognostically important data is missing, imputations will tend to be inaccurate and robustness will suffer. Accurate value imputation can be a complex issue since there are a wide variety of subjects that can influence the outcomes of individual studies in which a model is used, while factors like recruiting channels or study logistics can additionally affect the resulting study population cross-section.

### SUMMARY OF THE INVENTION

Systems and methods to account for uncertainties from missing covariates in generative model predictions in accordance with embodiments of the invention are illustrated. One embodiment includes a method that receives a set of known baseline data, that is substantially missing subject information on one or more covariates that could predictably impact an outcome of a model prediction. The method imputes various values for the one or more covariates with the set of known baseline data to create experimental data sets, determining estimated explained and unexplained variances in outcome for each subject, given the experimental data sets. The method utilizes the estimated explained and unexplained variances in outcome for each subject to derive an estimate for general variance in outcome for a population given the known baseline data and to define uncertainty in a generative model based on the estimate for general variance in outcome for a population given the known baseline data.

In a further embodiment, the estimate for general variance in outcome for a population given the set of known baseline data is evaluated using the following expression:

$$Var ( Y \vert {X}_{k n o w n} ) = \frac{1}{{n}^{2}} {\sum }_{i} ( {Δ}_{e x p , i}^{2} + {Δ}_{u n e x p , i}^{2} ) + α ( \frac{1}{{n}^{2}} ) [ {( {\sum }_{i} {Δ}_{u n e x p , i} )}^{2} - {\sum }_{i} {Δ}_{u n e x p , i}^{2} ]$$

whererin Y is an outcome for a population; X is the set of known baseline data; n is the number of subjects in the population; Δ_{exp,i} is the explained variance in outcome for subject i; Δ_{unexp,i} is the unexplained variance in outcome for subject i; and α is the correlation coefficient uniformly selected for the set of known baseline data.

In a further embodiment, the estimated explained variance in outcome for a particular subject is evaluated by, for each experimental data set, imputing the experimental data set into a predictive model; running a plurality of simulations with the predictive model; and deriving a value for mean in predicted outcome over the plurality of simulations. The evaluation also includes computation of the variance over all values for mean in predicted outcome.

In a yet further embodiment, the estimated unexplained variance in outcome for a particular subject is evaluated by, for each experimental data set, imputing the experimental data set into a predictive model; running a plurality of simulations with the predictive model; and deriving a value for variance in predicted outcome over the plurality of simulations. The evaluation also includes computation of the average over each value for variance in predicted outcome.

In a yet further embodiment, generative predictive models are applied to create predictions.

In another embodiment, unless imputed values for missing baseline data do not fully account for correlation between subjects, such as where all subjects have systematically higher or lower values of covariates, a default assumption for a given generative predictive model will be that variance contributions from subjects are uncorrelated. Another default assumption for a given generative predictive model will be that the estimated unexplained variance equals zero.

In still another embodiment, an updated covariance matrix, listing updated covariance values for every combination of subjects, will be established from combining covariance matrices in the following expression:

$$( \begin{matrix}{Δ}_{exp , 1}^{2} & 0 & … & 0 \\ 0 & {Δ}_{exp , 2}^{2} & … & 0 \\ & & … & \\ 0 & 0 & … & {Δ}_{exp , n}^{2}\end{matrix} ) + ( \begin{matrix}{Δ}_{unexp , 1}^{2} & {Δ}_{unexp , 1} {Δ}_{unexp , 2} {α}_{12} & … & {Δ}_{unexp , 1} {Δ}_{unexp , n} {α}_{1 n} \\ {Δ}_{unexp , 1} {Δ}_{unexp , 2} {α}_{12} & {Δ}_{unexp , 2}^{2} & … & {Δ}_{unexp , 2} {Δ}_{unexp , n} {α}_{2 n} \\ & & … & \\ {Δ}_{unexp , 1} {Δ}_{unexp , n} {α}_{1 n} & {Δ}_{unexp , 2} {Δ}_{unexp , n} {α}_{2 n} & … & {Δ}_{unexp , n}^{2}\end{matrix} )$$

wherein, Y is an outcome for a population; X is the set of known baseline data; n is the number of subjects in the population; Δ_{exp,i} is the explained variance in outcome for subject i; Δ_{unexp,i} is the unexplained variance in outcome for subject i; and α_{i,j} is the correlation coefficient for subjects i and j.

In a further embodiment, the general variance in outcome given the known baseline data will be determined from the following expression:

$$Var ( Y \vert {X}_{known} ) = \frac{1}{{n}^{2}} {\sum }_{i , j} ( {Cov}_{u p d} ( i , j ) )$$

wherein Cov_{upd}(i,j) is the updated covariance value for subjects i and j in the updated covariance matrix.

In another embodiment, the various values for the one or more covariates are imputed while having correlated values of uncertainty between them.

In another embodiment, the method further includes producing a quantitative estimate of a component of uncertainty derived from missing covariates by deriving a value for feature importance that assigns an absolute or relative weight to individual covariates from model-specific measures; and estimating the proportion of uncertainty due to missing covariates by using the feature importance.

One embodiment includes a non-transitory computer-readable medium including instructions which, when executed by a computer, cause the computer to carry out a process including receiving a set of known baseline data that that is substantially missing subject information on one or more covariates that could predictably impact an outcome of a model prediction; combining values for the one or more covariates with the set of known baseline data to create an experimental data set; determining an estimated explained and unexplained variance in an outcome for each subject, given the experimental data set; utilizing the estimated explained and unexplained variance in the outcome for each subject to derive an estimate for general variance in outcome for a population given the known baseline data; and define uncertainty in a generative model based on the estimate for general variance in outcome for a population given the known baseline data.

In a further embodiment, the estimate for general variance in outcome for a population given the set of known baseline data is evaluated using the following expression:

$$Var ( Y \vert {X}_{k n o w n} ) = \frac{1}{{n}^{2}} {\sum }_{i} ( {Δ}_{e x p , i}^{2} + {Δ}_{u n e x p , i}^{2} ) + α ( \frac{1}{{n}^{2}} ) [ {( {\sum }_{i} {Δ}_{u n e x p , i} )}^{2} - {\sum }_{i} {Δ}_{u n e x p , i}^{2} ]$$

wherein, Y is an outcome for a population; X is the set of known baseline data; n is the number of subjects in the population; Δ_{exp,i} is the explained variance in outcome for subject i; Δ_{unexp,j} is the unexplained variance in outcome for subject j; and α is the correlation coefficient uniformly selected for the set of known baseline data.

In a further embodiment, the estimated explained variance in outcome for a particular subject is evaluated by, for each experimental data set, imputing the experimental data set into a predictive model; running a plurality of simulations with the predictive model; and deriving a value for mean in predicted outcome over the plurality of simulations. The evaluation also includes computation of the variance over all values for mean in predicted outcome.

In a yet further embodiment, the estimated unexplained variance in outcome for a particular subject is evaluated by, for each experimental data set, imputing the experimental data set into a predictive model; running a plurality of simulations with the predictive model; and deriving a value for variance in predicted outcome over the plurality of simulations. The evaluation also includes computation of the average over each value for variance in predicted outcome.

In a still further embodiment, unless imputed values for missing baseline data do not fully account for correlation between subjects, such as where all subjects have systematically higher or lower values of covariates: a default assumption for a given generative predictive model will be that variance contributions from subjects are uncorrelated; and another default assumption for a given generative predictive model will be that the estimated unexplained variance equals zero.

In another embodiment, an updated covariance matrix, listing updated covariance values for every combination of subjects, will be established from combining covariance matrices in the following expression:

$$( \begin{matrix}{Δ}_{exp , 1}^{2} & 0 & … & 0 \\ 0 & {Δ}_{exp , 2}^{2} & … & 0 \\ & & … & \\ 0 & 0 & … & {Δ}_{exp , n}^{2}\end{matrix} ) + ( \begin{matrix}{Δ}_{unexp , 1}^{2} & {Δ}_{unexp , 1} {Δ}_{unexp , 2} {α}_{12} & … & {Δ}_{unexp , 1} {Δ}_{unexp , n} {α}_{1 n} \\ {Δ}_{unexp , 1} {Δ}_{unexp , 2} {α}_{12} & {Δ}_{unexp , 2}^{2} & … & {Δ}_{unexp , 2} {Δ}_{unexp , n} {α}_{2 n} \\ & & … & \\ {Δ}_{unexp , 1} {Δ}_{unexp , n} {α}_{1 n} & {Δ}_{unexp , 2} {Δ}_{unexp , n} {α}_{2 n} & … & {Δ}_{unexp , n}^{2}\end{matrix} )$$

wherein, Y is an outcome for a population; X is the set of known baseline data; n is the number of subjects in the population; Δ_{exp,i} is the explained variance in outcome for subject i; Δ_{unexp,j} is the unexplained variance in outcome for subject j; and α_{i,j} is the correlation coefficient for subjects i and j.

In a further embodiment, the general variance in outcome given the known baseline data will be determined from the following expression:

$$Var ( Y \vert {X}_{known} ) = \frac{1}{{n}^{2}} {\sum }_{i , j} ( {Cov}_{u p d} ( i , j ) )$$

wherein Cov_{upd}(i,j) is the updated covariance value for subjects i and j in the updated covariance matrix.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 conceptually illustrates a process for accounting for uncertainties from missing covariates in generative model predictions in accordance with an embodiment of the invention.

FIG. 2 illustrates a flowchart reflecting a set of various data structures involved in the process accounting for uncertainties from missing covariates in accordance with an embodiment of the invention.

FIG. 3 conceptually illustrates a step-by-step mapping for the process accounting for uncertainties from missing covariates in generative model predictions in accordance with an embodiment of the invention.

FIG. 4 conceptually illustrates a process for deriving generative model uncertainty estimates from approximations for variance in accordance with an embodiment of the invention.

FIG. 5 illustrates a system that provides for training and utilizing a generative model in response to an uncertainty calculation on the impact of missing covariates in accordance with an embodiment of the invention.

FIG. 6 illustrates a modeling element as applied to training and utilizing a generative model in response to an uncertainty calculation in accordance with an embodiment of the invention.

FIG. 7 illustrates an uncertainty calculation application as applied to training and utilizing a generative model in accordance with an embodiment of the invention.

FIG. 8 illustrates a representation of the impact of methods, in accordance with the invention, on clinical data from studies in Alzheimer's Disease, wherein key prognostic variables are uniformly missing.

FIG. 9 illustrates a second representation of the impact of these methods on clinical data from studies in Alzheimer's Disease; however, only mildly prognostic variables were uniformly missing.

### DETAILED DESCRIPTION

Systems and methods in accordance with some embodiments of the invention can account for missing covariates in the context of generative model predictions. Predictive models that, given input covariates, are capable of predicting the expected outcome as well as the variance of possible outcomes may be referred to as ‘generative models,’ predictive models,' or ‘generative predictive models’ throughout this description. In creating generative predictive models, unknowns among the covariates input into said model are a common challenge that can come in two main forms. One such form, sporadic missingness, occurs when covariates are observed, but their distribution is inconsistent among samples, such that an individual sample's missing covariates may not necessarily be the same as another sample's missing covariates. The other form data gaps can take, uniform missingness, occurs when one or more covariates are not measured at all for the entirety of a subject population. Systems and methods in accordance with numerous embodiments of the invention are applicable to issues of uniform missingness as well as sporadic missingness.

Generative models are typically able to compensate for missing covariates through data imputations. Such imputations can be performed either as a precursor to making predictions (pre-imputation) or as part of the predictive process itself (predictive imputation). However, imputations tend to be used when the accuracy of the imputed covariates is not expected to have a substantial impact on the robustness of the resulting prediction. If the covariates are in fact prognostically important, then the impact on model prediction uncertainty is more substantial. As a result, when a disparity exists between known baseline data and outcome-determinant data, most pre-imputation or predictive imputation methods will have to be supplemented.

When the data-collection process for a population used in a predictive is biased relative to the data-collection process eventually used to train the predictive model, it is likely that the missing covariates in the population will have distributional differences (i.e., different mean, different variance, or other different shape parameters) compared to the training data. Any relationship between the measured and unmeasured covariates may therefore be insufficient to characterize those differences. If so, without details on the data-taking process and their impact on the unmeasured covariates, the generative model prediction uncertainty values will have to be more expansive to compensate for said differences. Processes in accordance with many embodiments of the invention can account for the impact of the missing covariates on model prediction uncertainty. In certain embodiments, processes can account for potential differences between the population for which a generative model was trained and the population for which a particular prediction is being made. Processes in accordance with certain embodiments of the invention can incorporate the preceding points to provide estimates on the uncertainty of the model predictions.

The law for total variance follows the following formula:

$$Var ( Y \vert X ) = E [ Var ( Y \vert X ) ] + Var ( E [ Y \vert X ] )$$

Applying conditional probability to this formula in the context of predictive modeling, for individual subject i, when some baseline data is known (X_{known}), and some baseline data is not and needs to be estimated (X_{missing}), variance in outcome (Y) can be depicted as:

$$Var ( {Y}_{i} \vert {X}_{k n o w n , i} ) = E [ Var ( {Y}_{i} \vert {X}_{k n o w n , i} , {X}_{m i s s i n g , i} ) \vert {X}_{k n o w n , i} ] + Var ( E [ {Y}_{i} \vert {X}_{k n o w n , i} , {X}_{m i s s i n g , i} ] \vert {X}_{k n o w n , i} )$$

The variance in outcome given known baseline data may also be referred to as ‘Var(Y|X)’ or ‘inter-cohort variance’ throughout this description.

The first term of this formula as applied to subject i can be considered the ‘explained variance’ (Δ_{exp,i}), while the second can be the ‘unexplained variance’ (Δ_{unexp,i}).

For systems in accordance with many embodiments of the invention, when values are imputed for missing covariates, the explained component of variance (or simply ‘estimated explained variance’) can be estimated as the mean, taken over separate imputations, of the variance of predicted outcomes. Conversely, for some such embodiments, the unexplained component of variance ('estimated unexplained variance') can be estimated as the variance over mean predictions of outcomes. Once both values have been determined, processes in accordance with some embodiments of the invention can update the relevant generative models with a corresponding uncertainty estimate.

In several embodiments, processes can combine estimates for the fraction of uncertainty resulting from missing covariates and estimates for the fraction of uncertainty due to potential covariate variability into a single quantitative measure of uncertainty, which can be reported as the additional uncertainty over pre-existing methods (e.g. pre-imputation). In some such embodiments, a set of pre-imputed values may be chosen that spans a reasonable range of distributions. Once the imputed values are used to make predictions, the process can convert the distribution of outcomes into a value for uncertainty.

In certain embodiments, determinations of estimated unexplained variance can be assumed to be correlated across samples, leading to a derivation of inter-cohort variance over the course of producing predictive models (e.g. predictive imputation).

In a number of embodiments, population samples that influence predictions can be assumed to have correlated values of uncertainty. Processes in accordance with a number of embodiments of the invention can alter the degree of correlation between the values of certainty (e.g., manually by the practitioner, automatically based on heuristics, etc.). Under such embodiments, lower correlations may account for a larger potential difference in baseline data to the population training the model, and a larger uncertainty by extension.

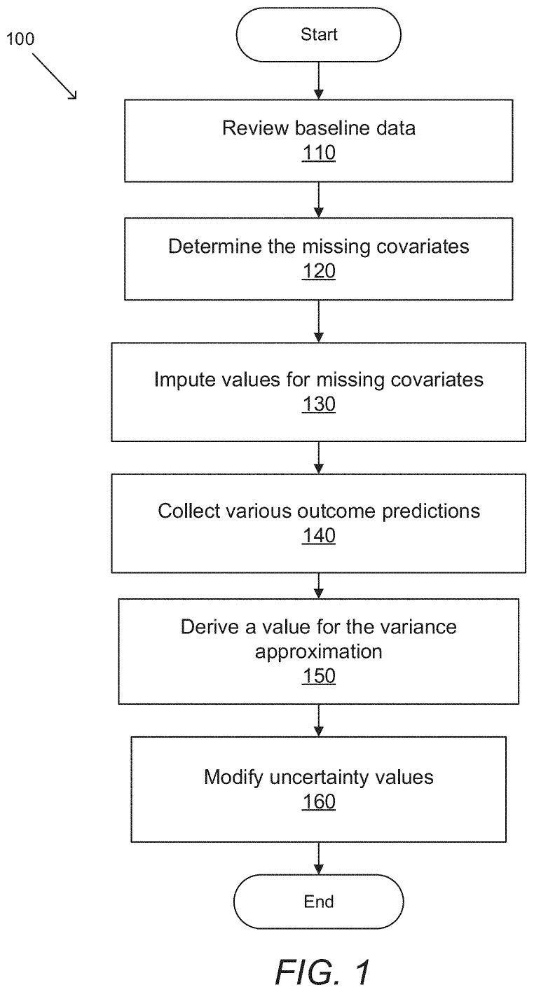

Turning now to the drawings, a flowchart depicting a process accounting for uncertainty, in accordance with some embodiments of the invention, is illustrated in FIG. 1. In this description, the terms tovariates' or ‘variables’ will refer to personal characteristics of the subjects in a research study (e.g. age, gender, CAT scan results), while baseline data refers to the data already available at the start of the study.

The process 100 reviews (110) baseline data, in order to determine the information likely to have a significant effect on a generative model's predicted outcome. A threshold for significance may vary according to factors including, but not limited to, the number of subjects associated with a given model and the total amount of covariates in consideration. In accordance with many embodiments of the invention, a review (110) of baseline data can include an analysis of all available baseline data for unavailable covariates with a substantial likelihood of significantly affecting the uncertainty of any generative models. Through the review, process 100 identifies covariates that are (1) prognostically important (likely to have a significant impact on the outcome that results); and (2) predominantly or entirely unaccounted for in the data set being used to train predictive models.

In accordance with many embodiments of the invention, the process can determine (120) the missing covariates among the baseline data collected on a set of subjects. For a covariate to be classified as ‘missing,’ every subject need not have unavailable baseline data on the covariate. For a given generative model, the majority of subjects not having available baseline data on the prognostically important covariate may be sufficient for a covariate to be considered missing.

Given a set of one or more missing covariates, the process 100, in accordance with many embodiments of the invention, may impute (130) values for missing covariates into the generative model, over the course of a plurality of predictive simulations. In this description, the terms ‘predictive simulation’ or ‘simulation’ may refer to a generative model being run with a particular set of modeling data until a predicted outcome is produced. In many embodiments of the invention, imputing a value for a missing covariate may include running the predictive simulation, wherein the known baseline data can be incorporated into the particular set of modeling data. In some such embodiments, the missing covariates can have their values estimated utilizing the values of other known covariates through a variety of methods including, but not limited to, estimates based on linear regression. Once the missing covariates are estimated, the process 100 may incorporate the missing covariate estimates into the particular set of modeling data.

The process 100, in accordance with many embodiments of the invention, may collect (140) various outcome predictions for imputed missing covariates running the plurality of predictive simulations. Collecting various outcome predictions, in accordance with a number of embodiments of the invention, can include running the plurality of predictive simulations to determine the resulting outcomes for generative models. In numerous embodiments, processes may collect a set of outcome predictions corresponding to the plurality of predictive simulations. In several embodiments, processes may maintain accounts of which imputed values for missing covariates correspond to which outcome predictions.

The process 100, through repeatedly imputing (130) of values for missing covariates and collecting (140) various outcome predictions, may derive (150) a value for variance approximation. The value for variance approximation may also be referred to as an approximation for ‘variance given the known baseline data’ or ‘inter-cohort variance.’ The process 100 may, in assessing the variance given the known baseline data, be able to modify (160) uncertainty values reflected in generative models utilizing the set of known baseline data.

In some embodiments, an alternative means of deriving uncertainty values may use ‘feature importance.’ Processes in accordance with such embodiments of the invention can evaluate feature importance by assigning absolute and/or relative weights to individual covariates (or ‘features’) on the basis of prognostic importance. In some such embodiments, measures of feature importance can be used to produce quantitative estimates of the component of uncertainty derived from missing covariates. Values for feature importance may be derived from correlation coefficients between the individual covariates and the outcome. Alternatively, feature importance may be derived from other model-specific measures.

In some such embodiments, estimates for total variability can also be derived from feature importance. An example of this is scaling the proportion of uncertainty by the coefficient of determination (R²) between each of the covariates and the outcome. A value for the proportion of uncertainty values that are due to missing covariates can similarly be estimated from a measurement of feature importance. For example, an estimate may be derived from standardized formulae like the magnitude of the missing covariates' feature importance over the total sum of the magnitudes of all feature importances.

In some embodiments, deriving a value for the portion of uncertainty attributed to missing covariates may use a dataset, referred to in this description as a ‘reference dataset.’ The reference dataset can store missing covariates that are used to estimate the portion of uncertainty attributed to said missing covariates. If the reference dataset relates to the population of interest, the reference dataset may produce a value for the proportion of uncertainty attributed to uniform missingness. Systems in accordance with some such embodiments may use a generative model to create predictions for the reference dataset, wherein missing covariates are alternatively present and set to be missing. A fractional reduction in the uncertainty in the predictions when the covariates are present can be assigned to the fraction of uncertainty for the population of interest, for cases including the missing covariates.

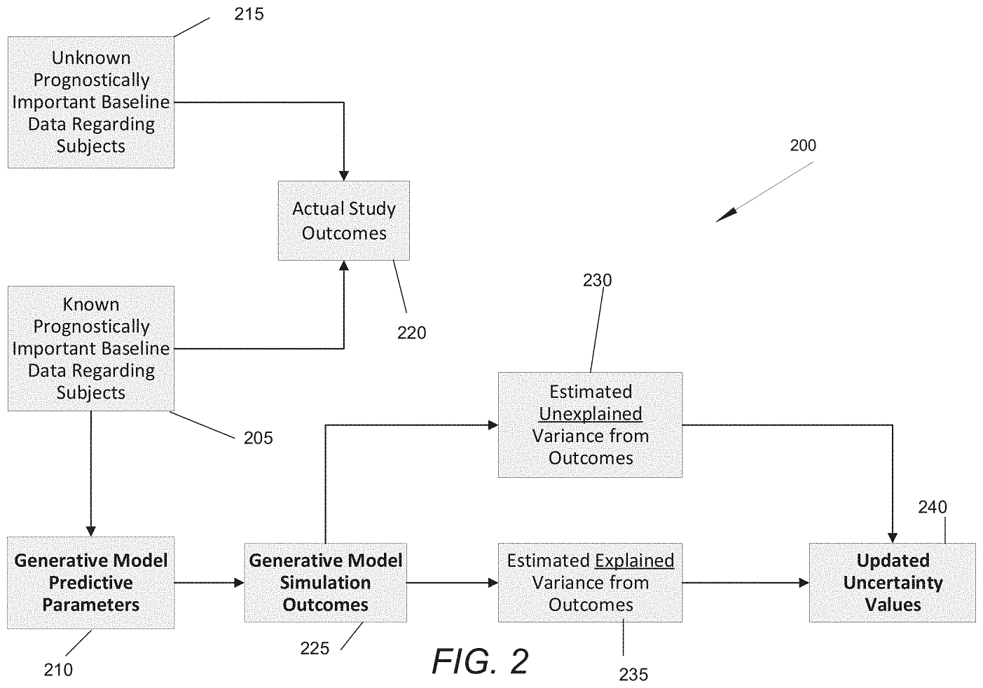

An example of a system 200 in accordance with many embodiments of the invention is illustrated in FIG. 2. As the makeup of a particular study population can be impacted by multiple factors, from the design and logistics of a study to the particular study sites and recruiting channels used, accurate imputation is especially important. Additionally, the degree of uncertainty or confidence for a given predictive model is heavily dependent on the breadth of data imputed into that model. Again, prognostically important baseline data encapsulates the covariates that are particularly likely to impact actual study outcomes 220. In determining the uncertainty associated with a generative model, the degree of prognostic importance that all baseline data possesses may be analyzed and classified. As such, baseline data may be divided into known prognostically important baseline data 205 and unknown prognostically important baseline data 215.

The first category of baseline data, known prognostically important baseline data 205, in accordance with a number of embodiments of the invention, can include data that is uniformly, or near-uniformly available for a given population. In collecting information, the more prognostically important information that is known about particular covariates, the more confident the eventual generative model will be. For example, in a model reflecting a study on cancer, collected information on the subjects' respective smoking histories are both known and likely to have a substantial impact on the actual study outcomes 220. In accordance with many embodiments of the invention, the known prognostically important baseline data 205 can have direct and quantifiable impact on the eventual generative model predictive parameters 210. That classification of baseline data is immediately accessible, so its impact on training the parameters of a particular model is direct.

Conversely, the second category, unknown prognostically important baseline data 215, can have indeterminate influence on a particular model. Unknown prognostically important baseline data 215 is data whose absence negatively impacts the confidence/uncertainty of a given prediction to a significant degree. For example, in the earlier hypothetical study, most participants may choose not to answer an optional question (e.g. what is your profession) the answer for which may substantially impact their likelihood to develop cancer. This data is also prognostically important, but the degree of its impact is unknown compared to the known prognostically important baseline data 215.

Regardless of whether prognostically important data is known or unknown, the data may impact the actual study outcomes 220. Therefore, in approximating actual study outcomes 220, systems in accordance with numerous embodiments of the invention can directly impute known prognostically important baseline data 205 into the current generative model predictive parameters 210.

For unknown prognostically important baseline data 215, to train the generative model predictive parameters 210, the unknown data may have to be estimated. However, since this data will likely be influenced by variety in the population used to train the model, accuracy in the estimates may substantially affect the overall certainty for the model. As mentioned, the degree of uncertainty or confidence for a given predictive model is heavily dependent on the breadth of data. Should a generative model not have access to data likely to impact the eventual outcome, while the prediction may not change, the certainty about the prediction can.

For systems in accordance with many embodiments of the invention, one may use a plurality of estimates for unknown prognostically important baseline data 215. In doing so, multiple possible values for the unknown prognostically important baseline data 215 can be individually imputed into the generative model predictive parameters 210 over the course of distinct simulations. Typically, a system using a generative model might obtain certainty in the form of variance, through running a plurality of simulations with a constant dataset. However, to account for unknown prognostically important baseline data 215, multiple imputations of the unknown data can be performed and the values recorded. In those cases, imputing multiple values may provide a similar opportunity to observe the breadth of a particular covariate's impact on the model observed.

This process can be performed through using a plurality of simulations under the generative model predictive parameters 210, to obtain various generative model simulation outcomes 225. To refine the results associated with the imputed values, a plurality of simulations may be performed for each individual imputation of unknown prognostically important baseline data 215. In incorporating the eventual outcomes of the plurality simulations into a valid estimate for uncertainty, two important values may be needed.

Generative model simulation outcomes 225 in accordance with certain embodiments of the invention can be used to determine a value for estimated explained variance from outcomes 235. Broadly speaking, estimated explained variance may refer to the variance between groups. For instances where multiple different values for unknown prognostically important baseline data 215 are imputed, estimated explained variance may assess the expected value for variance in results across separate imputations (i.e., E[ Var(Y|X_{Known}) ]).

Second, simulation outcomes can also be used to determine estimated unexplained variance from outcomes 230. Broadly speaking, unexplained variance may refer to the variance within a group. For instances where multiple different values for unknown prognostically important baseline data 215 are imputed, unexplained variance may assess the variance in the expected results across separate imputations (i.e., Var( E[Y|X_{Known}])).

Once combined, estimates of explained and unexplained variances obtained from simulation outcomes can be used to produce a value for variance in outcome given the known baseline data. This is the value that can correspond to updated uncertainty values 240 which account for the impact that unknown baseline data is likely to have on actual study outcomes 220.

As mentioned, a value for variance in outcome given the known baseline data may be used in order to obtain updated uncertainty values 240. In accordance with many embodiments of the invention, the variance in outcome given the known baseline data may be estimated as generally applied to a population from a set of estimates for explained and unexplained variance. In doing so, the system may predict the updated variance using the following formula:

$$Var ( Y \vert {X}_{k n o w n} ) = \frac{1}{{n}^{2}} [ {\sum }_{i} {Δ}_{e x p , i}^{2} + ( 1 - α ) {\sum }_{i} {Δ}_{u n e x p , i}^{2} + {α ( {\sum }_{i} {Δ}_{u n e x p , i} )}^{2} ] = \frac{1}{{n}^{2}} {\sum }_{i} {Δ}_{e x p , i}^{2} + {Δ}_{u n e x p , i}^{2} + α ( \frac{1}{{n}^{2}} ) [ {( {\sum }_{i} {Δ}_{unexp , i} )}^{2} - {\sum }_{i} {Δ}_{u n e x p , i}^{2} ]$$

where n may be the number of subjects in the population; Δ_{exp,i} the explained variance in outcome for subject i; and Δ_{unexp,i} the unexplained variance in outcome for subject i.

In some such embodiments of the invention, under an assumption of negligible difference for covariances within a population, the form of the correlation coefficient may be represented by α. The correlation coefficient can be a value that may be imputed and constant for all subjects. The default values for a correlation coefficient imputation may include various values, such as (but not limited to) 0, 0.1, 0.5, or 1. The correlation coefficient value can be imputed so as to account for the concern that the imputed distributions, X_{missing}, do not fully account for correlations between subjects. Determinations in accordance with such embodiments of the invention may also assume that the explained component of variance for each subject is uncorrelated, and therefore not incorporate α, as shown above. Meanwhile the unexplained component can be correlated among subjects under such embodiments.

Alternatively, in accordance with many embodiments of the invention, deriving the variance in the outcome (Y) given the known baseline data (X_{known}) may follow the following formula:

$$Var ( Y \vert {X}_{known} ) = \frac{1}{{n}^{2}} {\sum }_{i , j} Cov ( i , j )$$

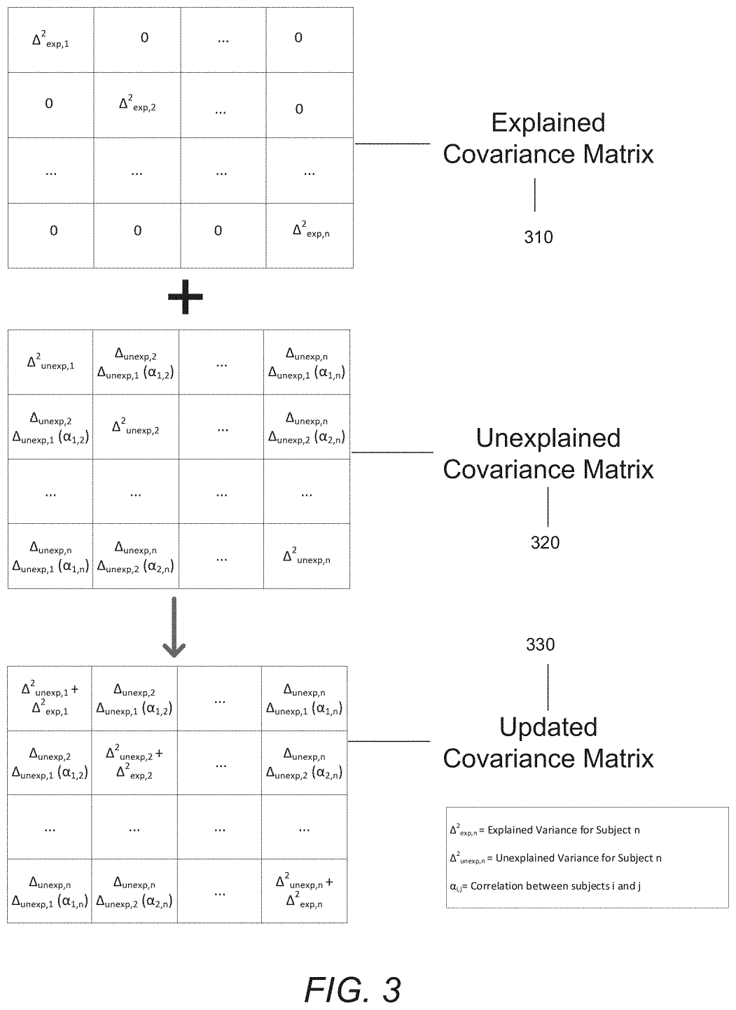

which may represent the mean of all terms in the covariance matrix across subjects, scaled by the number of subjects (n). In certain embodiments of the invention, illustrated in FIG. 3, the combination of explained and unexplained variances may be represented in matrices. Under those circumstances, the values used to determine the variance in outcome given the known baseline data, can be derived through an entity that this description may refer to as the updated covariance matrix 330.

Processes in accordance with some embodiments of the invention can account for covariance between subjects, rather than using the explained and unexplained variance in isolation. In accordance with many embodiments of the invention, when using the prior formula, the updated covariance matrix may depict all the covariance values needed to determine the variance in outcome given X_{known}. As the updated covariance matrix may also account for the covariance between each pair of subjects, it may even provide a more specific estimate of variance in outcome given known baseline data. Finally, deriving values for the updated covariance matrix may be done through the combination of two constituent matrices.

One such constituent matrix is the explained covariance matrix 310, which depicts the explained components of variance (or covariance) for each subject (or pair of subjects). Covariance matrices in accordance with numerous embodiments of the invention are square matrices providing the covariance between each pair of subjects. For this matrix, the diagonal can depict the explained variance for each subject (since, for row X and column X in a covariance matrix, Coy (X,X)=Var(X)). However, in accordance with many embodiments of the invention, the default assumption can be that the explained component of variance for each subject is uncorrelated. Given this assumption, only the explained variance for each subject may be accounted for, and there can be an absence in assumed covariance between two different subjects. As a result, explained covariance matrices in accordance with many embodiments of the invention may be in the form of diagonal matrices where the only nonzero values are the respective explained variance estimates for each subject.

Meanwhile, unexplained covariance matrices 320 in accordance with various embodiments of the invention can depict the unexplained components of variance. Therefore, the diagonal of the unexplained covariance matrix 320 may list the unexplained component of variance for each subject. Unlike the explained variance matrix, the default assumption is that the unexplained component of variance may be correlated between subjects. Therefore, every non-diagonal value in the matrix may still show the unexplained component of covariance between the subjects corresponding to the rows and columns, respectively (e.g. ‘row X, column Y’ and ‘row Y, column X’ illustrate the unexplained component of covariance between subjects X and Y). As is the case for normal covariance, the elements corresponding to subjects X and Y may follow the following format in determining the unexplained component of covariance:

$${Cov}_{unexp} ( X , Y ) = {p}_{x , y} * {Δ}_{u n e xp , x} * {Δ}_{u n e x p , y}$$

wherein ρ_{x,y} is the correlation coefficient between subjects x and y; Δ_{unexp,x} is the square-root of the unexplained component of variance for subject x; and Δ_{unexp,y}, the square-root of the unexplained component of variance for subject y.

The combination of the explained 310 and unexplained 320 covariance matrices may produce the updated covariance matrix 330 as illustrated in FIG. 3. As mentioned above, updated covariance matrices in accordance with various embodiments of the invention can be used to determine the variance in outcome via the mean of all terms in the covariance matrix across subjects, scaled by the number of subjects (n). This value, in many embodiments of the invention, can correspond to the optimal uncertainty range.

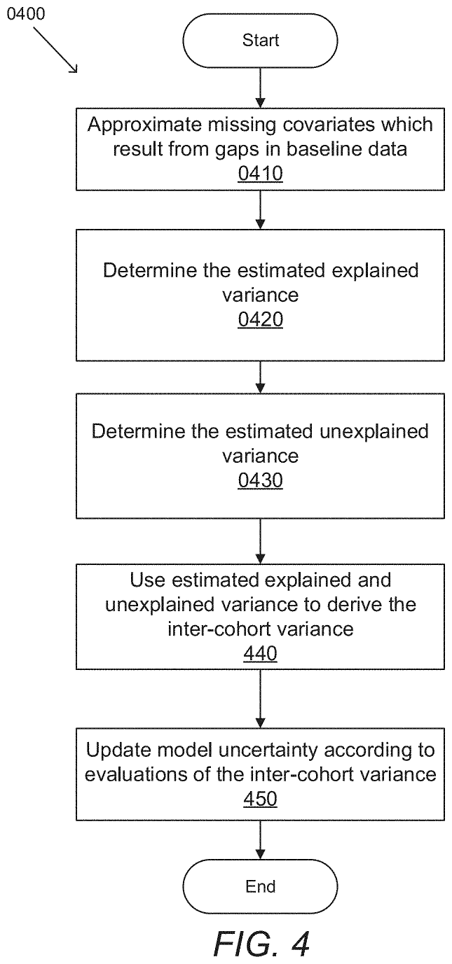

An example of a process 400 of running a plurality of simulations to obtain respective estimates for the explained and unexplained variance is illustrated in FIG. 4. By imputing baseline data sets into individual simulations, the process 400 can approximate (410) missing covariates which result from gaps in baseline data. In establishing a plurality of baseline data sets, a plurality of approximations for unknown baseline data may be used. Just as the imputed values are used to make predictions, the distribution of outcomes may be used to assign uncertainty values.

In accordance with many embodiments of the invention, uncertainty may be obtained through estimating the explained and unexplained variance. Determining (420) the estimated explained variance may include determining the average, over different imputations, of outcome variance, over simulations, when imputing baseline data sets. This can include deriving outcome variance over multiple simulations for a singular imputation and/or determining the average outcome variance after multiple different imputations have run.

For the particular set of modeling data that corresponds to a singular imputation, deriving outcome variance over simulations may comprise the imputation undergoing a plurality of simulations without adjusting the values of the missing covariates. In many embodiments of the invention, the plurality of simulations may number at least ten. Given the plurality of simulations, the output may be a plurality of outcomes. Outcome variance over simulations may then refer to the variance over the plurality of outcomes.

Determining the average over ‘different imputations’ may refer to multiple different values being imputed for the missing covariates. In many embodiments of the invention, the number of different imputations may number at least ten. Given a set of different imputations, each imputation running a plurality of simulations (as mentioned in the prior step), the output may be a set of values for outcome variance, each corresponding to a singular imputation. Determining the average over different imputations may then refer to the average of the set of values for outcome variance. Through determining the average of all the values for outcome variance, processes in accordance with many embodiments of the invention may then produce the estimate for explained variance.

Determining (430) the estimated unexplained variance can utilize similar steps under many embodiments of the invention. When imputing baseline data sets, determining (430) the estimated unexplained variance may include determining the variance, over different imputations, of average outcome, over simulations. This can also include deriving average outcome over multiple simulations for a singular imputation and determining the variance over the average outcomes from multiple different imputations.

For the particular set of modeling data that corresponds to a singular imputation, deriving the average outcome over simulations may comprise the imputation undergoing a plurality of simulations without adjusting values of the missing covariates. In many embodiments of the invention, the plurality of simulations may number at least ten. Given a plurality of simulations, the output may be a plurality of outcomes. Average outcome over simulations may then refer to the average of the plurality of outcomes.

Determining the variance over ‘different imputations’ may refer to different values being imputed for the missing covariates. Given a set of different imputations, each imputation running a plurality of simulations (as mentioned in the prior step), the output may be a set of values for outcome variance, each corresponding to a singular imputation. Determining the variance over different imputations may then refer to the variance over the set of values for average outcome.

Through determining the variance over the set of values for average outcome, the process 400 may then determine (430) the estimated unexplained variance. In many embodiments of the invention, the process 400 may use (440) estimated explained and unexplained variance to derive an estimate the inter-cohort variance, for example, through the following formula:

$$Var ( Y \vert {X}_{k n o w n} ) = \frac{1}{{n}^{2}} [ {\sum }_{i} {Δ}_{e x p , i}^{2} + ( 1 - α ) {\sum }_{i} {Δ}_{u n e x p , i}^{2} + {α ( {\sum }_{i} {Δ}_{u n e x p , i} )}^{2} ] = \frac{1}{{n}^{2}} {\sum }_{i} {Δ}_{e x p , i}^{2} + {Δ}_{u n e x p , i}^{2} + α ( \frac{1}{{n}^{2}} ) [ {( {\sum }_{i} {Δ}_{unexp , i} )}^{2} - {\sum }_{i} {Δ}_{u n e x p , i}^{2} ]$$

Given the inter-cohort-variance, the process 400 can update (450) model uncertainty according to evaluations of the inter-cohort variance. In particular, a derivation of inter-cohort variance can map directly to the values for uncertainty associated with a generative model (when using the particular set of known baseline data). Once a value for the uncertainty (under the particular set of known baseline data) has been procured, the process 400 may input this value into future generative models that use the same set of known baseline data.

While specific processes for accounting for uncertainty in a generative predictive model are described above, any of a variety of processes can be utilized to establish uncertainty values as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted. Although the above embodiments of the invention are described in reference to the utilization of variance to estimate uncertainty, the techniques disclosed herein may be used in any type of uncertainty modification.

### A. Systems for Modifying Generative Models

### 1. Model Modification System

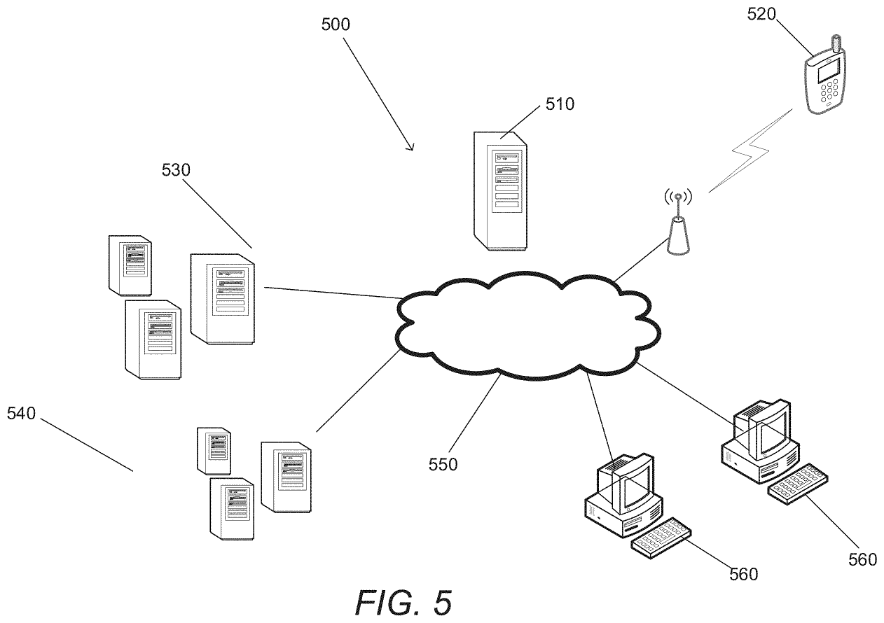

A system that provides for the modification of models and modeling datasets, as well as the generation of predictive models in accordance with some embodiments of the invention, is illustrated in FIG. 5. Network 500 includes a communications network 550. The communications network 550 is a network such as the Internet that allows devices connected to the network 550 to communicate with other connected devices. Server systems 510, 530, and 540 are connected to the network. Each of the server systems 510, 530, and 540 is a group of one or more servers communicatively connected to one another via internal networks that execute processes that provide cloud services to users over the network. However, the server systems 510, 530 and 540 may include any number of servers and any additional number of server systems may be connected to the network 550.

One skilled in the art will recognize that a model modification system may exclude certain components and/or include other components that are omitted for brevity without departing from the invention. For purposes of this discussion, cloud services are one or more applications that are executed by one or more server systems to provide data and/or executable applications to devices over a network. The server systems 510, 530 and 540 are shown each having three servers in the internal network 550. However, the server systems may include any number of servers and any additional number of server systems may be connected to the network to provide cloud services. In accordance with various embodiments of the invention, a network that uses systems and methods that create generative models in accordance with an embodiment of the invention may be provided by a process (or a set of processes) being executed on a single server system and/or a group of server systems communicating over network.

Various functions (e.g., data processing, data collection, statistical analysis, uncertainty derivation, etc.) of modeling elements in accordance with some embodiments can be implemented on a single processor, on multiple cores of a single computer, and/or distributed across multiple processors on multiple different computers. Similarly, various storages (e.g., data processing, data collection, statistical analysis, uncertainty derivation, etc.) of data collection and modification systems in accordance with several embodiments can be stored in a single database, distributed across multiple database servers, or distributed across multiple different database platforms on multiple different servers.

Users may use personal devices 520, 560 that connect to the network to perform processes for providing and/or interaction with a network that uses systems and methods that create generative models in accordance with various embodiments of the invention. In the shown embodiment, the personal devices 560 are shown as desktop computers that are connected via a conventional “wired” connection to the network 550. However, the personal device 560 may be a desktop computer, a laptop computer, a smart television, an entertainment gaming console, or any other device that connects to the network 550 via a “wired” connection. The mobile device connects to network using a wireless connection. A wireless connection is a connection that uses Radio Frequency (RF) signals, Infrared signals, or any other form of wireless signaling to connect to the network. In FIG. 5, the mobile device is a mobile telephone. However, mobile device 520 may be a mobile phone, Personal Digital Assistant (PDA), a tablet, a smartphone, or any other type of device that connects to network via wireless connection without departing from the invention.

As can readily be appreciated, the specific computing system used to construct models from data is largely dependent upon the requirements of a given application and should not be considered as limited to any specific computing system(s) implementation.

### 2. Modeling Element

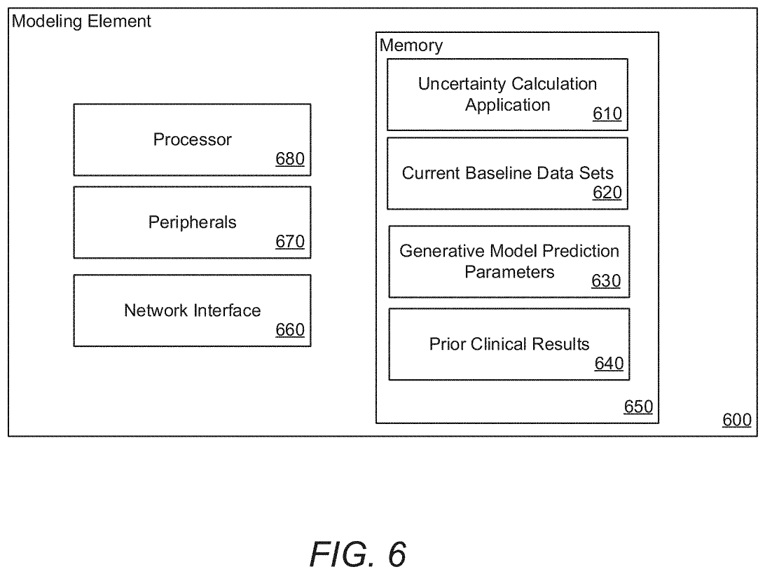

An example of a modeling element 600 that executes instructions to perform processes that utilizes current baseline data sets 620 to update generative model prediction parameters 630 and supplement prior clinical results 640 in accordance with various embodiments is shown in FIG. 6. Modeling elements in accordance with many embodiments can include (but are not limited to) one or more of mobile devices, servers, cloud services, and/or other computers. Modeling element 600 includes processor 680, peripherals 670, network interface 660, and memory 650. One skilled in the art will recognize that a modeling element may exclude certain components and/or include other components that are omitted for brevity without departing from the invention.

The processor 680 can include (but is not limited to) a processor, microprocessor, controller, or a combination of processors, microprocessor, and/or controllers that performs instructions stored in the memory to manipulate data stored in the memory 650. Processor instructions can configure the processor to perform processes in accordance with certain embodiments.

Peripherals 670 can include any of a variety of components for capturing data, such as (but not limited to) cameras, displays, and/or sensors. In a variety of embodiments, peripherals can be used to gather inputs and/or provide outputs. Modeling element 600 can utilize network interface to transmit and receive data over a network based upon the instructions performed by processor 680. Peripherals 670 and/or network interfaces 660 in accordance with many embodiments of the invention can be used to gather data that can be used to update generative model prediction parameters 630.

Memory 650 includes a collection of current baseline data sets 620, generative model prediction parameters, and a collection of prior clinical results 640. Current baseline data sets 620 and prior clinical results 640, in accordance with many embodiments of the invention can be used to pre-train generative models, such as digital twins, to generate potential outcomes. In numerous embodiments, current baseline data sets 620 can include (but is not limited to) patient registries, electronic health records, and/or real-world data. In many embodiments, predictions from a generative model can be compared to new studies that were not used to train the model in order to compare how predictions generalize to new populations.

Network interfaces 660 in accordance with a variety of embodiments can be used for various functions, such as (but not limited to) interacting with datasets, communicating across a network, receiving user inputs, and/or providing notifications based upon the instructions performed by processor.

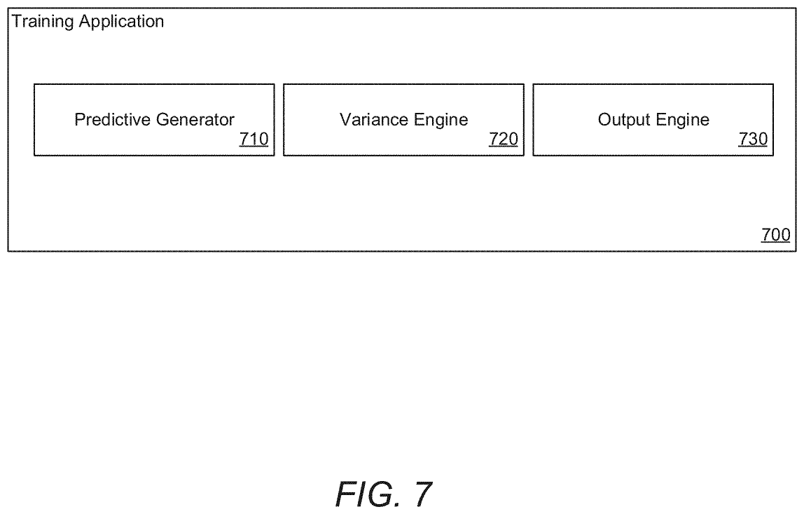

Memory also includes uncertainty calculation application 610, described below and illustrated in FIG. 7. Uncertainty calculation applications in accordance with several embodiments can be used to convert current baseline data sets 620 into uncertainty values to input into generative model prediction parameters. One skilled in the art will recognize that an uncertainty calculation application 610 may exclude certain components and/or include other components that are omitted for brevity without departing from the invention.

Although a specific example of a modeling element 600 is illustrated in FIG. 6, any of a variety of modeling elements can be utilized to perform processes for constructing models from data similar to those described herein as appropriate to the requirements of specific applications in accordance with embodiments.

### 3. Uncertainty Calculation Application

An example of an uncertainty calculation application for estimating uncertainty ranges in accordance with an embodiment of the invention is illustrated in FIG. 7. Uncertainty calculation application 700 includes predictive generator 710, variance function 720, and output engine 730. One skilled in the art will recognize that an uncertainty calculation application may exclude certain components and/or include other components that are omitted for brevity without departing from the invention.

Predictive generators 710 in accordance with various embodiments of the invention can produce generative predictive models including, but not limited to, digital twin models. Generative predictive models in accordance with certain embodiments of the invention can generate potential outcome data based on characteristics of an individual and/or a population. The data used by a predictive generator 710 in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. In some embodiments, predictive generators, as applied to digital twins, can be used to simulate patient populations, disease progressions, and/or predicted responses to various treatments.

Variance engines 720 in accordance with several embodiments of the invention can be used to derive approximations for variance in outcome given known baseline data. Variance engines 720 may perform this derivation in response to the outcomes produced by a given generative predictive model. This derivation can incorporate data analytics including, estimated explained variance, estimated unexplained variance, construction of explained covariance matrices, construction of unexplained covariance matrices, and construction of updated covariance matrices. Variance engines 720 in accordance with several embodiments of the invention can convert the aforementioned values and matrices into approximations for variance in outcome given known baseline data.

Output engines 730 in accordance with several embodiments of the invention can provide a variety of outputs to a user, including (but not limited to) generative model biases, model responses, recommended study designs, etc. In numerous embodiments, output engines 730 can provide feedback when the results of generative predictive models diverge from actual study outcomes. For example, output engines 730 in accordance with certain embodiments of the invention can provide a notification when a difference between generated control outcomes for subjects and their actual control outcomes exceeds a threshold. Alternatively, output engines can provide feedback on the efficiency of updated certainty calculations. For example, output engines 730 in accordance with certain embodiments of the invention can provide a notification when values for updated uncertainty exceed a threshold.

Although a specific example of an uncertainty calculation application is illustrated in FIG. 7, any uncertainty calculation applications can be utilized to convert current baseline data sets into uncertainty values similar to those described herein, as appropriate to the requirements of specific applications in accordance with embodiments of the invention.

### B. Implementation of the Invention

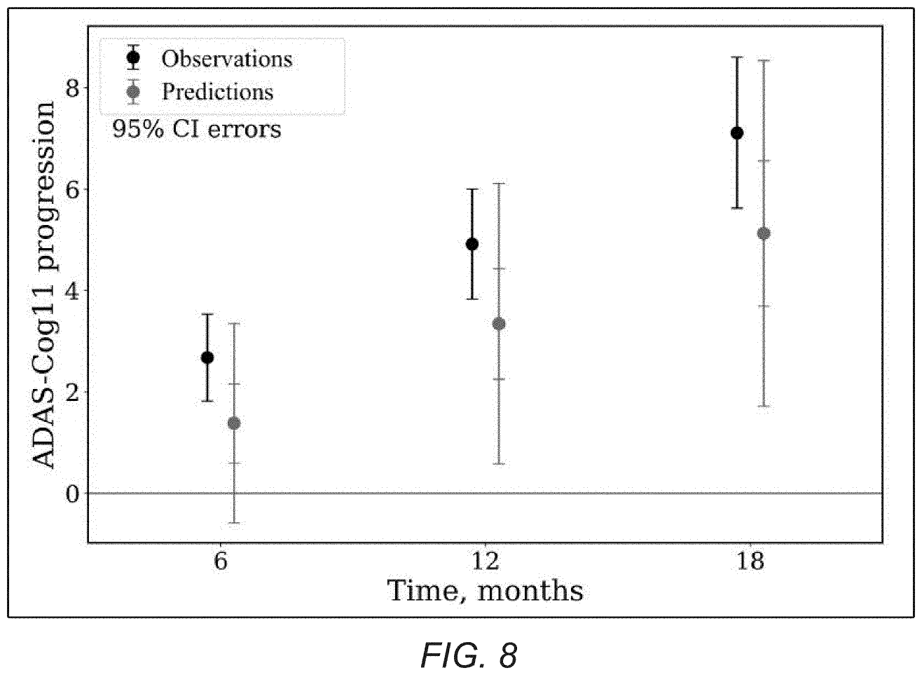

FIG. 8 illustrates an example of the impact that methods operating in accordance with the invention may have on representations of updated generative models where key prognostic variables are uniformly missing. Specifically, this figure depicts application of some such methods to clinical data from studies in Alzheimer's Disease. The relevant study depicts various follow-up times (6 months, 12 months, and 18 months, respectively). In the resulting graph, black points show the observed data while black error bars show the 95% confidence interval from statistical uncertainties. Meanwhile, the data in blue reflects those conclusions determined through predictive models: blue points show mean predictions and the inner blue error bars show the 95% confidence interval uncertainty from the generative model's predictions when uniform missingness is addressed only through predictive imputation. Finally, the outer blue error bars reflect a 95% confidence interval derived, in compliance with several embodiments of the invention, through formulae for updated uncertainty values. For the purposes of this figure, the imputed correlation coefficient (a) is set to 0.5.

As might be evident from FIG. 8, there is a substantial increase in the uncertainty accounted for when the present method is used (the outer blue bars). Most significantly, said increased uncertainties bring outcome predictions into better statistical agreement with the observed results (the black bars) than the original uncertainties from predictive imputation (the inner blue bars), regardless of the follow-up time.

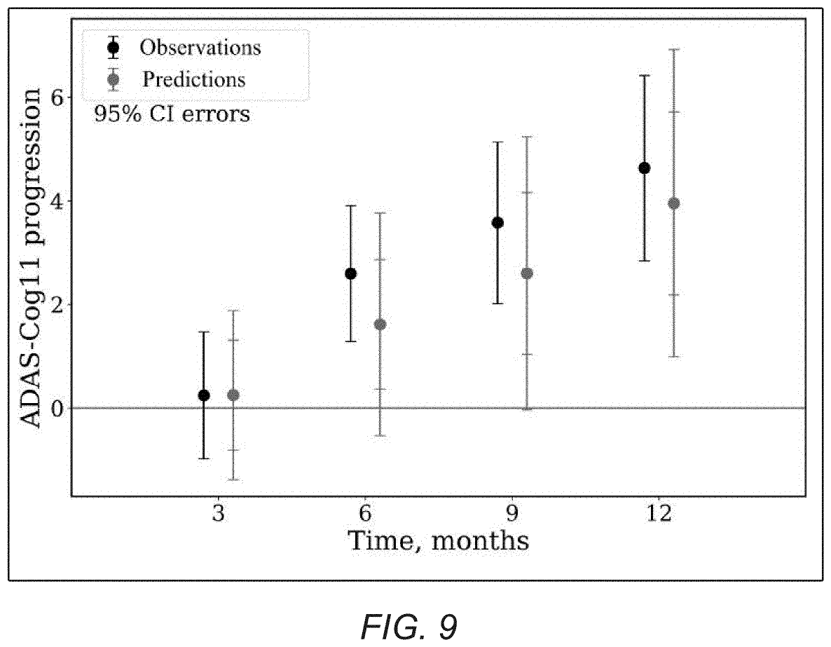

FIG. 9 illustrates another example of the impact that methods operating in accordance with the invention may have on representations of updated generative models, although as applied to instances where uniformly missing covariates have only mild prognostic variables. This figure depicts application of some such methods to clinical data from a separate Alzheimer's Disease study, in which follow-up times were 3 months, 6 months, 12 months, and 18 months, respectively. In FIG. 9, black points again reflect the observed data; black error bars show the 95% confidence interval from statistical uncertainties; blue points show mean predictions; the inner blue error bars show the 95% confidence interval uncertainty from the generative model's predictions when uniform missingness is addressed only through predictive imputation; and the outer blue error bars show the 95% confidence interval using the present invention's formula for inter-cohort variance and an imputed correlation coefficient of 0.5.

Compared to FIG. 8, when the present method is used there is less of an adjustment in uncertainty. However, the increased uncertainties again bring the outcome predictions into better statistical agreement with the observed results (regardless of the follow-up time). As is the case in FIG. 8, this suggests that systems acting in accordance with many embodiments of the invention have increased capacity to compensate for gaps in baseline data when predicting uncertainty.

Although specific methods of accounting for uncertainties from missing covariates are discussed above, many different methods of generative predictive model analysis can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for defining uncertainty in generative predictive models, the method comprising:
receiving a set of known baseline data that is substantially missing subject information on one or more covariates that could predictably impact an outcome of a model prediction;
imputing various values for the one or more covariates with the set of known baseline data to create experimental data sets;
determining an estimated explained variance in an outcome for each subject, given the experimental data sets;
determining an estimated unexplained variance in the outcome for each subject given the experimental data sets;
utilizing the estimated explained variance in the outcome for each subject and the estimated unexplained variance in the outcome for each subject to derive an estimate for general variance in outcome for a population given the known baseline data; and
defining uncertainty in a generative model based on the estimate for general variance in outcome for a population given the known baseline data.

2. The method of claim 1, wherein the estimate for general variance in outcome for a population given the set of known baseline data is evaluated using the following expression:
$$Var ( Y \vert {X}_{k n o w n} ) = \frac{1}{{n}^{2}} {\sum }_{i} ( {Δ}_{e x p , i}^{2} + {Δ}_{u n e x p , i}^{2} ) + α ( \frac{1}{{n}^{2}} ) [ {( {\sum }_{i} {Δ}_{u n e x p , i} )}^{2} - {\sum }_{i} {Δ}_{u n e x p , i}^{2} ]$$
whererin,
Y is an outcome for a population;
X is the set of known baseline data;
n is the number of subjects in the population;
Δ_{exp,i} is the explained variance in outcome for subject i;
Δ_{unexp,i} is the unexplained variance in outcome for subject i; and
α is the correlation coefficient uniformly selected for the set of known baseline data.

3. The method of claim 2, wherein the estimated explained variance in outcome for a particular subject is evaluated using the following process:
for each experimental data set:
imputing the experimental data set into a predictive model;
running a plurality of simulations with the predictive model; and
deriving a value for mean in predicted outcome over the plurality of simulations; and
computing variance over all values for mean in predicted outcome.

4. The method of claim 3, wherein the estimated unexplained variance in outcome for a particular subject is evaluated using the following process:
for each experimental data set:
imputing the experimental data set into a predictive model;
running a plurality of simulations with the predictive model; and
deriving a value for variance in predicted outcome over the plurality of simulations; and
computing an average over each value for variance in predicted outcome.

5. The method of claim 4, wherein generative predictive models are applied to create predictions.

6. The method of claim 1, wherein, unless imputed values for missing baseline data do not fully account for correlation between subjects, such as where all subjects have systematically higher or lower values of covariates:
a default assumption for a given generative predictive model will be that variance contributions from subjects are uncorrelated; and
another default assumption for a given generative predictive model will be that the estimated unexplained variance equals zero.

7. The method of claim 1, wherein an updated covariance matrix, listing updated covariance values for every combination of subjects, will be established from combining covariance matrices in the following expression:
$$( \begin{matrix}{Δ}_{exp , 1}^{2} & 0 & … & 0 \\ 0 & {Δ}_{exp , 2}^{2} & … & 0 \\ & & … & \\ 0 & 0 & … & {Δ}_{exp , n}^{2}\end{matrix} ) + ( \begin{matrix}{Δ}_{unexp , 1}^{2} & {Δ}_{unexp , 1} {Δ}_{unexp , 2} {α}_{12} & … & {Δ}_{unexp , 1} {Δ}_{unexp , n} {α}_{1 n} \\ {Δ}_{unexp , 1} {Δ}_{unexp , 2} {α}_{12} & {Δ}_{unexp , 2}^{2} & … & {Δ}_{unexp , 2} {Δ}_{unexp , n} {α}_{2 n} \\ & & … & \\ {Δ}_{unexp , 1} {Δ}_{unexp , n} {α}_{1 n} & {Δ}_{unexp , 2} {Δ}_{unexp , n} {α}_{2 n} & … & {Δ}_{unexp , n}^{2}\end{matrix} )$$
wherein,
Y is an outcome for a population;
X is the set of known baseline data;
n is the number of subjects in the population;
Δ_{exp,i} is the explained variance in outcome for subject i;
Δ_{unexp,i} is the unexplained variance in outcome for subject i; and
α_{i,j} is the correlation coefficient for subjects i and j.

8. The method of claim 7, wherein the general variance in outcome given the known baseline data will be determined from the following expression:
$$Var ( Y \vert {X}_{known} ) = \frac{1}{{n}^{2}} {\sum }_{i , j} ( {Cov}_{u p d} ( i , j ) )$$
wherein Cov_{upd}(i,j) is the updated covariance value for subjects i and j in the updated covariance matrix.

9. The method of claim 1, wherein the various values for the one or more covariates are imputed while having correlated values of uncertainty between them.

10. The method of claim 1, further comprising: producing a quantitative estimate of a component of uncertainty derived from missing covariates by:
deriving a value for feature importance that assigns an absolute or relative weight to individual covariates from model-specific measures; and
estimating the proportion of uncertainty due to missing covariates by using the feature importance.

11. A non-transitory computer-readable medium comprising instructions which, when executed by a computer, cause the computer to carry out a process comprising:
receiving a set of known baseline data that that is substantially missing subject information on one or more covariates that could predictably impact an outcome of a model prediction;
combining values for the one or more covariates with the set of known baseline data to create an experimental data set;
determining an estimated explained variance in an outcome for each subject, given the experimental data set;
determining an estimated unexplained variance in the outcome for each subject given the experimental data set;
utilizing the estimated explained variance in the outcome for each subject and the estimated unexplained variance in the outcome for each subject to derive an estimate for general variance in outcome for a population given the known baseline data; and
defining uncertainty in a generative model based on the estimate for general variance in outcome for a population given the known baseline data.

12. The non-transitory computer-readable medium of claim 11, wherein the estimate for general variance in outcome for a population given the set of known baseline data is evaluated using the following expression:
$$Var ( Y \vert {X}_{known} ) = \frac{1}{{n}^{2}} {Σ}_{i} ( {Δ}_{exp , i}^{2} + {Δ}_{unexp , i}^{2} ) + α ( \frac{1}{{n}^{2}} ) [ {( {Σ}_{i} {Δ}_{unexp , i} )}^{2} - {Σ}_{i} {Δ}_{unexp , i}^{2} ]$$
wherein,
Y is an outcome for a population;
X is the set of known baseline data;
n is the number of subjects in the population;
Δ_{exp,i} is the explained variance in outcome for subject i;
Δ_{unexp,j} is the unexplained variance in outcome for subject j; and
α is the correlation coefficient uniformly selected for the set of known baseline data.

13. The non-transitory computer-readable medium of claim 12, wherein the estimated explained variance in outcome for a particular subject, further comprising:
for each experimental data set:
imputing the experimental data set into a predictive model;
running a plurality of simulations with the predictive model; and
deriving a value for mean in predicted outcome over the plurality of simulations; and
computing variance over all values for mean in predicted outcome.

14. The non-transitory computer-readable medium of claim 13, wherein the estimated unexplained variance in outcome for a particular subject, further comprising:
for each experimental data set:
imputing the experimental data set into a predictive model;
running a plurality of simulations with the predictive model; and
deriving a value for variance in predicted outcome over the plurality of simulations; and
computing an average over each value for variance in predicted outcome.

15. The non-transitory computer-readable medium of claim 14, wherein, unless imputed values for missing baseline data do not fully account for correlation between subjects, such as where all subjects have systematically higher or lower values of covariates:
a default assumption for a given generative predictive model will be that variance contributions from subjects are uncorrelated; and
another default assumption for a given generative predictive model will be that the estimated unexplained variance equals zero.

16. The non-transitory computer-readable medium of claim 12, wherein an updated covariance matrix, listing updated covariance values for every combination of subjects, will be established from combining covariance matrices in the following expression:
$$( \begin{matrix}{Δ}_{exp , 1}^{2} & 0 & ⋯ & 0 \\ 0 & {Δ}_{exp , 2}^{2} & ⋯ & 0 \\ & & ⋯ & \\ 0 & 0 & ⋯ & {Δ}_{exp , n}^{2}\end{matrix} ) + ( \begin{matrix}{Δ}_{unexp , 1}^{2} & {Δ}_{unexp , 1} {Δ}_{unexp , 2} {α}_{12} & ⋯ & {Δ}_{unexp , 1} {Δ}_{unexp , n} {α}_{1 n} \\ {Δ}_{unexp , 1} {Δ}_{unexp , 2} {α}_{12} & {Δ}_{unexp , 2}^{2} & ⋯ & {Δ}_{unexp .2} {Δ}_{unexp , n} {α}_{2 n} \\ & & ⋯ & \\ {Δ}_{unexp , 1} {Δ}_{unexp , n} {α}_{1 n} & {Δ}_{unexp , 2} {Δ}_{unexp , n} {α}_{2 n} & ⋯ & {Δ}_{unexp , n}^{2}\end{matrix} )$$
wherein,
Y is an outcome for a population;
X is the set of known baseline data;
n is the number of subjects in the population;
Δ_{exp,i} is the explained variance in outcome for subject i;
Δ_{unexp,j} is the unexplained variance in outcome for subject j; and
α_{i,j} is the correlation coefficient for subjects i and j.

17. The non-transitory computer-readable medium of claim 16, wherein the general variance in outcome given the known baseline data will be determined from the following expression:
$$Var ( Y \vert {X}_{known} ) = \frac{1}{{n}^{2}} {Σ}_{i , j} ( {Cov}_{upd} ( i , j ) )$$
wherein Cov_{upd}(i,j) is the updated covariance value for subjects i and j in the updated covariance matrix.
